(19) United States
(12) Patent Application Publication
(10) Pub. No.: US 2024/0085557 A1
(43) Pub. Date: Mar. 14, 2024

(54) COHERENT MATRIX OF DIGITAL IMAGING SYSTEMS ON CHIP

(71) Applicant: Exo Imaging, Inc., Santa Clara, CA (US)

(72) Inventors: Kutay USTUNER, Mountain View, CA (US); Dongwoon HYUN, Santa Clara, CA (US)

(21) Appl. No.: 18/243,608

(22) Filed: Sep. 7, 2023

Related U.S. Application Data
(60) Provisional application No. 63/375,097, filed on Sep. 9, 2022.

(51) Int. Cl. G01S 15/89; G01S 7/52

(52) U.S. Cl. CPC G01S 15/8927; G01S 7/52028; G01S 7/5205; G01S 15/8918; G01S 15/892; G01S 15/8925

(57) ABSTRACT

Provided herein are systems, devices, and methods for ultrasound imaging particular to matrix arrays of ultrasound transducer assemblies which each comprise a matrix array of transducer elements and an ASIC coupled to the matrix array of transducer elements. The matrix array of ultrasound transducer assemblies can be assembled into a variety of form factors. Virtual elements located in gaps between transducer assemblies may be defined. Synthesized receive signals may be generated for these virtual elements.

### CROSS-REFERENCE TO RELATED APPLICATIONS

This patent application claims the benefit of U.S. Provisional Application No. 63/375,097, filed Sep. 9, 2022, which is incorporated herein by reference in its entirety.

### BACKGROUND

The present disclosure relates to systems, devices, and methods for ultrasound imaging, particularly three-dimensional (3D) imaging.

The following patent references may be relevant: U.S. Pat. Pub. Nos. 2021/0183832, 2021/0028792, 2020/0405271, 2020/0405267, 2020/0405266, 2020/0315586, 2019/0361102, 2019/0299251, 2019/0261954, 2019/0261955, 2018/0366102, 2018/0361431, 2019/0196012, 2019/0212424, 2019/0133556, 2016/0151045, 2019/0388059, 2015/0297193, 2017/0135676, 2016/0202349, 2016/0242739, 2017/0296144, 2017/0296145, 2014/0243676, 2012/0143059, 2010/0249596, 2009/0326375, 2009/0240152, 2007/0016023, 2009/0007414, 2005/0068221, and 2001/0020130, as well as U.S. Pat. Nos. 10,755,692, 10,857,567, 11,154,276, 10,641,879, 10,405,829, 9,592,032, 9,521,991, 9,439,625, 8,545,406, 8,416,643, 8,926,514, 8,834,369, 8,137,280, 6,937,176, 5,928,152, 5,675,554, 5,685,308, 5,555,534, and 5,970,025.

### SUMMARY

The systems, methods and devices of this disclosure each have several innovative aspects, no single one of which is solely responsible for the desirable attributes disclosed herein.

The present disclosure relates to systems, devices, and methods for ultrasound imaging, in particular 3D imaging with massive numbers of transducer elements.

The present disclosure provides systems, devices, and methods for a full-array digital 3D transmit and receive beamformer that can be integrated on an application specific integrated circuit (ASIC), which in turn can be integrated on a high element count two-dimensional (2D) array transducer. This may reduce cost, size, weight, and power of an ultrasound imaging system. In turn, these high-element-count 2D array transducers can be assembled into a variety of form factors for a variety of use cases.

Aspects of the present disclosure provide methods for ultrasound beam forming and imaging with a plurality of ultrasound transducer assemblies, each ultrasound transducer assembly comprising a plurality of transducer elements. An exemplary method may comprise the steps of: (i) adjusting element coordinates of each transducer element for relative tilt and offset of each transducer assembly with respect to a common coordinate system; (ii) computing transmit delays and weights for each transducer element based on the adjusted element coordinates and transmit focus angle and depth; (iii) transmitting a pulse and receiving an echo from an object being imaged; (iv) processing receive signals of each transducer element; (v) synthesizing receive signals for one or more virtual elements within one or more gaps between the ultrasound transducer assemblies; and (vi) forming a dynamically focused receive beam based on the processed receive signals of the one or more transducer elements and synthesized receive signals of the one or more virtual elements.

In some embodiments, step (iv) comprises amplifying the receive signals of each transducer element and digitizing the amplified receive signal of each transducer element.

In some embodiments, step (v) comprises defining virtual elements for the one or more gaps between the ultrasound transducer assemblies and generating the synthesized receive signals for the virtual elements using the processed receive signals of the one or more transducer elements. In some embodiments, generating the synthesized receive signal of an individual virtual element comprises identifying a nearest transducer element to said individual virtual element and assigning the processed receive signal from said individual element as the synthesized receive signal of said individual virtual element. In some embodiments, generating the synthesized receive signal of an individual virtual element comprises identifying a first nearest transducer element on a first ultrasound transducer assembly on a first side of an individual gap, identifying a second nearest transducer element on a second transducer assembly on a second side of the individual gap opposite the first side, generating a linear interpolation of the processed receive signals of the first and second nearest transducer elements, and assigning said linear interpolation as the synthesized receive signal of said individual virtual element.

In some embodiments, step (vi) comprises: (a) computing delay(s) and weight(s) for each transducer element and virtual element based on the adjusted element coordinates and receive angle and focal depth, (b) applying the delays and weights on the amplified and digitized receive signals of the one or more transducer elements and on the synthesized receive signals of the one or more virtual elements, and (c) summing the delayed and weighted receive signals of all transducer elements of the plurality of ultrasound transducer assemblies and the virtual elements to form the dynamically focused receive beam.

In some embodiments, the steps (iv) to (vi) are repeated for the same receive beam line of sight but using the echo received in response to a plurality of transmit beams with foci that are laterally distinct, and the receive beams formed are time aligned and coherently summed to form synthesized receive beams.

In some embodiments, an application specific integrated circuit (ASIC) is integrated with at least one ultrasound transducer assembly, and the ASIC performs one or more of steps (i) to (vi) to form the dynamically focused receive beam.

In some embodiments, at least one ultrasound transducer assembly of the plurality is comprised of one or more capacitive micromachined ultrasound transducer (cMUT), piezoelectric micromachined ultrasound transducer (pMUT), or bulk PZT transducer elements.

In some embodiments, the plurality of ultrasound transducer assemblies comprises a matrix or array of the ultrasound transducer assemblies. In some embodiments, the matrix or array of the ultrasound transducer assemblies comprises a 1-dimensional array, a 2-dimensional matrix, a curved matrix or array, a piece-wise curved matrix or array, or a flat matrix or array of the ultrasound transducer assemblies.

In some embodiments, the plurality of the transducer elements for at least one ultrasound transducer assembly comprises a 2-dimensional matrix of the transducer elements.

In some embodiments, the plurality of ultrasound transducer assemblies is provided on a wearable device.

Further aspects of the present disclosure provide methods of imaging a target object.

An exemplary method may comprise using an imaging device to generate an image of the target object, wherein the imaging device comprises a plurality of ultrasound transducer assemblies and control circuitry operatively coupled thereto, and wherein the control circuitry is configured to operate the plurality of the ultrasound transducer assemblies according to any of the methods described herein.

Another exemplary method may comprise providing an imaging device to generate an image of the target object, wherein the imaging device comprises a plurality of ultrasound transducer assemblies and control circuitry operatively coupled thereto, and wherein the control circuitry is configured to operate the plurality of the ultrasound transducer assemblies according to any of the methods described herein.

Another exemplary method may comprise the steps of providing a plurality of ultrasound transducer assemblies, each ultrasound transducer assembly comprising a plurality of ultrasound transducer elements; tiling the plurality of ultrasound transducer assemblies into a matrix configuration; and acquiring an image of the target object using the tiled plurality of ultrasound transducer assemblies, wherein the plurality of ultrasound transducer assemblies is operatively coupled to control circuitry configured to operate the plurality of ultrasound transducer assemblies according to any of the methods described herein. In some embodiments, tiling the plurality of ultrasound transducer assemblies comprises arranging the plurality of ultrasound transducer assemblies into a 1-dimensional array, a 2-dimensional matrix or array, a curved matrix or array, a piece-wise curved matrix or array, or a flat matrix or array.

Further aspects of the present disclosure provide systems of imaging a target object. An exemplary system may comprise: a plurality of ultrasound transducer assemblies, each ultrasound transducer assembly comprises a plurality of transducer elements; and control circuitry operatively coupled to the plurality of ultrasound transducer assemblies and configured to operate the plurality of ultrasound transducer assemblies according to any of the methods described herein. In some embodiments, the ultrasound transducer assemblies are tileable into a matrix configuration. In some embodiments, the matrix configuration is a 1-dimensional array, a 2-dimensional matrix or array, a curved matrix or array, a piece-wise curved matrix or array, or a flat matrix or array.

Further aspects of the present disclosure provide methods of imaging a target object. An exemplary method may comprise the steps of: providing a plurality of ultrasound transducer assemblies, each ultrasound transducer assembly comprising a plurality of ultrasound transducer elements; tiling the plurality of ultrasound transducer assemblies into a matrix or array configuration; and acquiring an image of the target object using the tiled plurality of ultrasound transducer assemblies.

In some embodiments, the matrix or array configuration is a 1-dimensional array, a 2-dimensional matrix or array, a curved matrix array, a piece-wise curved matrix or array, or a flat matrix or array.

In some embodiments, each ultrasound transducer assembly further comprises an application specific integrated circuit (ASIC) integrated thereon.

In some embodiments, each ultrasound transducer assembly of the plurality is adjusted for relative tilt and offset with respect to a common coordinate system for the plurality of ultrasound transducer assemblies.

Further aspects of the present disclosure provide systems of imaging a target object. An exemplary system may comprise: (a) a plurality of ultrasound transducer assemblies, each ultrasound transducer assembly comprises a plurality of transducer elements; and (b) control circuitry operatively coupled to the plurality of ultrasound transducer assemblies and configured to operate the plurality of ultrasound transducer assemblies, wherein the ultrasound transducer assemblies are tileable into a matrix or array configuration.

In some embodiments, the matrix or array configuration is a 1-dimensional array, a 2-dimensional matrix or array, a curved matrix or array, a piece-wise curved matrix or array, or a flat matrix or array.

In some embodiments, one or more gaps are present between adjacent ultrasound transducer assemblies when tiled into the matrix or array configuration.

In some embodiments, each ultrasound transducer assembly comprises an application specific integrated circuit (ASIC) operatively coupled to and integrated with the plurality of transducer assemblies for each ultrasound transducer assembly.

In some embodiments, each ultrasound transducer assembly of the plurality is adjusted for relative tilt and offset with respect to a common coordinate system for the plurality of ultrasound transducer assemblies.

In some embodiments, at least one ultrasound transducer assembly of the plurality is comprised of one or more capacitive micromachined ultrasound transducer (cMUT), piezoelectric micromachined ultrasound transducer (pMUT), or bulk PZT transducer elements.

In some embodiments, the plurality of the transducer elements for at least one ultrasound transducer assembly comprises a matrix or array of the transducer elements.

In some embodiments, the plurality of the transducer elements for at least one ultrasound transducer assembly comprises a 2-dimensional matrix of the transducer elements.

In some embodiments, the exemplary system further comprises a wearable housing configured to hold the plurality of ultrasound transducer assemblies in the matrix or array configuration.

In some embodiments, the wearable housing is a patch or band.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 shows an exemplary schematic diagram of an ultrasonic system using a transducer assembly comprised of a 2D array of transducers and an ASIC mounted on a PCB with additional circuitry, and a remote processor with a user interface and display, according to some embodiments.

FIG. 2 shows exemplary form factors for one or more tileable ultrasound transducer assemblies, according to some embodiments.

FIG. 3A shows a graph of a one-way aperture function of an exemplary 131-ultrasound transducer assembly configuration with no gaps (unbroken line), a 131-ultrasound transducer assembly configuration with a three-element gap (spaced apart broken line), a 35-ultrasound transducer assembly configuration with a three element gap (dotted line), and a 11-ultrasound transducer assembly configuration with a three element gap (closely packed broken line), according to some embodiments.

FIG. 3B shows a graph of a one-way lateral response of the exemplary 131-ultrasound transducer assembly configuration with no gaps (unbroken line), a 131-ultrasound transducer assembly configuration with a three-element gap (spaced apart broken line), a 35-ultrasound transducer assembly configuration with a three element gap (dotted line), and a 11-ultrasound transducer assembly configuration with a three element gap (closely packed broken line), according to some embodiments.

FIG. 4A shows a graph of a two-way aperture function of the exemplary 131-ultrasound transducer assembly configuration with no gaps (unbroken line), a 131-ultrasound transducer assembly configuration with a three-element gap (spaced apart broken line), a 35-ultrasound transducer assembly configuration with a three element gap (dotted line), and a 11-ultrasound transducer assembly configuration with a three element gap (closely packed broken line), according to some embodiments.

FIG. 4B shows a graph of a two-way lateral response of the exemplary 131-ultrasound transducer assembly configuration with no gaps (unbroken line), a 131-ultrasound transducer assembly configuration with a three-element gap (spaced apart broken line), a 35-ultrasound transducer assembly configuration with a three element gap (dotted line), and a 11-ultrasound transducer assembly configuration with a three element gap (closely packed broken line), according to some embodiments.

FIG. 5A shows a flow chart of an exemplary method of ultrasound beam forming and ultrasound imaging with a plurality of tileable ultrasound transducer assemblies, according to some embodiments.

FIG. 5B shows a flow chart further breaking down the step of forming a dynamically focused receive beam based on the processed receive signals and synthesized receive signals, according to some embodiments.

FIG. 6 shows a graph of a lateral response of an exemplary 128-ultrasound transducer assembly configuration array with 4 missing middle ultrasound transducer assemblies, according to some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Unless otherwise defined, all technical terms used herein have the same meaning as commonly understood by one of ordinary skill in the art to which the present subject matter belongs.

### Ultrasound Imaging System

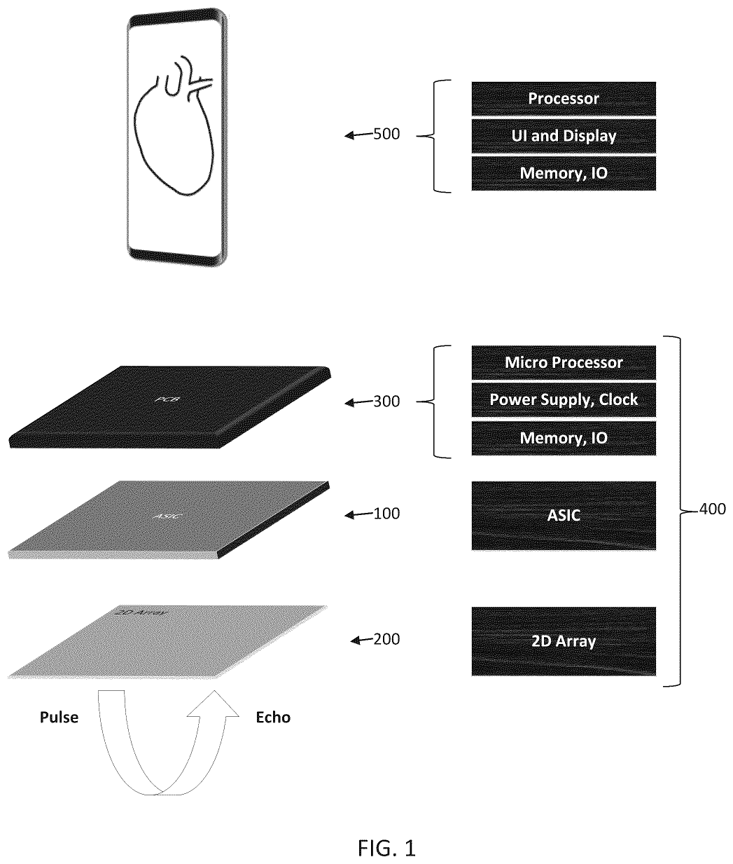

FIG. 1 shows an exemplary embodiment of the ultrasonic imaging system disclosed herein. The imaging system may include an ASIC 100 preferably integrated with a transducer 200. The transducer may be a one-dimensional or a two-dimensional array of pMUT (piezoelectric micromachined ultrasound transducer), cMUT (capacitive micromachined ultrasound transducer), or bulk PZT elements. The ASIC and transducer array are typically mounted on a PCB (or PCBs) 300. The PCB may have additional circuitry such as a microprocessor, power supply (battery, regulators), clock, memory and/or an input output device.

The ASIC, transducer array, and the PCB form a transducer assembly 400. The area of the transducer assembly may match the area of the transducer array to keep the footprint small. The transducer assembly can be packaged in a patch, or in a wearable or holdable housing.

The transducer assembly, via an input output device, may communicate with a remote processor 500 that may include a user interface, display and memory. The processor may be a mobile device such as a smart phone, smart watch, pad, or a laptop, or it can be a desktop computer. It may perform image processing, perform plane and volume rendering, and connect to a network and database such as electronic health records. The communication between the transducer assembly and the remote processor may be wired or wireless, using standard communication protocols.

The microprocessor on the transducer assembly may initialize the ASIC with a small set of parameters such as the imaging frequency and the transmit and receive f-numbers and then may provide the transmit and receive beam parameters (beam origin, angle, focus depth) for each pulse-echo (transmit-receive) event in the scan sequence. An on-ASIC delay and weight computer may compute the transmit and receive beamforming parameters (delay and weight) for each beam defined by the transmit and receive beam parameters. The ASIC may send out a steered and focused transmit pulse, may receive the echo from tissue at each transducer element, and may form receive beams using ASIC-computed delay and weight. The output of the ASIC is typically the fully formed beams using the full aperture.

Ultrasound transducer assemblies comprising a matrix array of transducer elements and an ASIC operatively coupled to such a matrix array are described in PCT Application No. PCT/US2022/011417, filed Jan. 6, 2022, and U.S. patent application Ser. No. 17/569,805, filed Jan. 6, 2022, which are incorporated herein by reference.

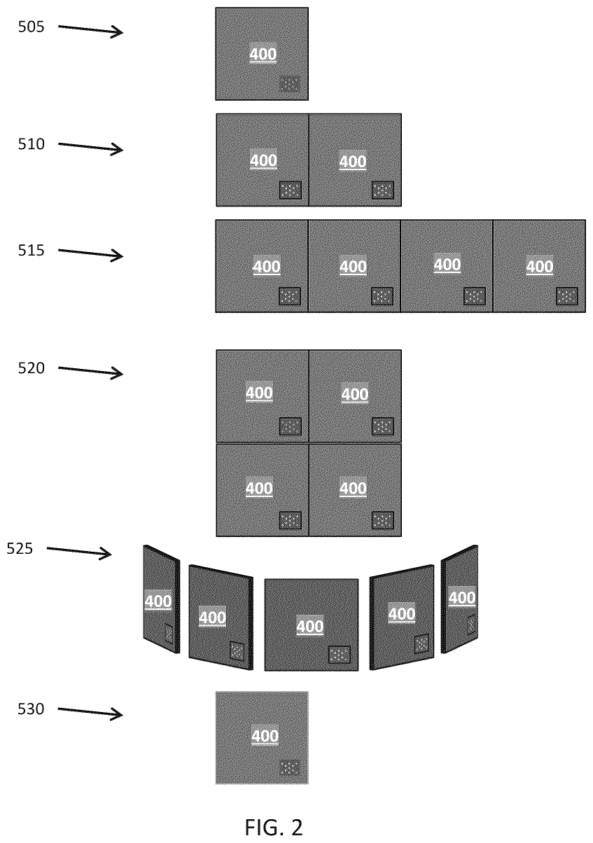

Another aspect provided herein, per FIG. 2, is a system for imaging a target object. In some embodiments, the system comprises: a plurality of ultrasound transducer assemblies 400, each ultrasound transducer assembly 400 comprising a plurality of transducer elements; and control circuitry operatively coupled to the plurality of ultrasound transducer assemblies 400 and configured to operate the plurality of ultrasound transducer assemblies 400, wherein the ultrasound transducer assemblies 400 are tileable into a matrix configuration. In some embodiments, each ultrasound transducer assembly 400 comprises a plurality of transducers (e.g., 64×64, 64×24, 48×24). In some embodiments, the tileable and modular capabilities of the ultrasound transducer assemblies 400 herein enable their formation matrices of variable acoustic window size and performance. In some embodiments, the acoustic window size is an area through which a patient's body can be imaged.

In some embodiments, as acoustic window size varies among the clinical applications such as, for example, pediatric, cardiac, abdomen, obstetrics, vascular, breast tomography and high-intensity focused ultrasound (HIFU), various array sizes are required for each application. As such, FIG. 2 illustrates that the ultrasound transducer assembly 400 is tileable as a single assembly or multiple assemblies in one or more straight or piecewise curved dimensions to enable its use for applications with various window size requirements.

In some embodiments, the transducer assembly 400 is tileable into a variety of array or matrix configurations. In some embodiments, the matrix configuration is a 1-dimensional array, a 2-dimensional array or matrix, a curved array or matrix, or a flat array or matrix. In some embodiments, the array or matrix configuration comprises a standalone configuration 505, a one-dimensional array configuration 510 or 515, a two-dimensional matrix configuration 520, a curved one-dimensional array configuration 525, or a curved two-dimensional matrix configuration. The one-dimensional array configurations may comprise one-dimensional array configuration 510 with two assemblies or one-dimensional array configuration 515 with four assemblies.

In some embodiments, the matrix configuration 505 provides advantages in, for example, wearable ultrasound devices or patches, vasculature, abdominal, or pulmonary imaging devices, or surgical guide devices, to name a few. In some embodiments, one-dimensional matrix configurations 510, 515 provide advantages in, for example, cardiac, abdomen, breast, and vascular imaging applications, to name a few. In some embodiments, two-dimensional matrix configurations 520 provides advantages in, for example high-intensity focused ultrasound (HIFU) applications. In some embodiments, a curved one-dimensional matrix configuration 525, or a curved two-dimensional matrix configuration provides advantages in, for example, tomography. In some embodiments, a modified standalone matrix configuration 530 provides advantages for intracardiac echocardiography (ICE).

In some embodiments, one or more gaps are present between adjacent ultrasound transducer assemblies 400 when tiled into the matrix configuration. In some embodiments, each ultrasound transducer assembly 400 comprises an application specific integrated circuit (ASIC) operatively coupled to and integrated with the plurality of transducer assemblies 400 for each ultrasound transducer assembly 400. In some embodiments, at least one ultrasound transducer assembly 400 of the plurality is comprised of one or more capacitive micromachined ultrasound transducer (cMUT), piezoelectric micromachined ultrasound transducer (pMUT), or bulk PZT transducer elements. In some embodiments, the plurality of the transducer elements for at least one ultrasound transducer assembly 400 comprises a matrix array of the transducer elements.

In some embodiments, the system further comprises a wearable housing configured to hold the plurality of ultrasound transducer assemblies 400 in the matrix configuration. In some embodiments, the wearable housing is a patch or band.

### Method for Ultrasound Beam Forming and Imaging

In some embodiments, manufacturing matrix configurations that maintain zero gaps between transducer assemblies is difficult and cost prohibitive due to manufacturing tolerances and capabilities. However, these gaps can create discontinuities in the aperture function when a gap is in the active transmit and/or receive aperture, which can increase side lobes and therefore increase acoustic clutter and reduce contrast resolution.

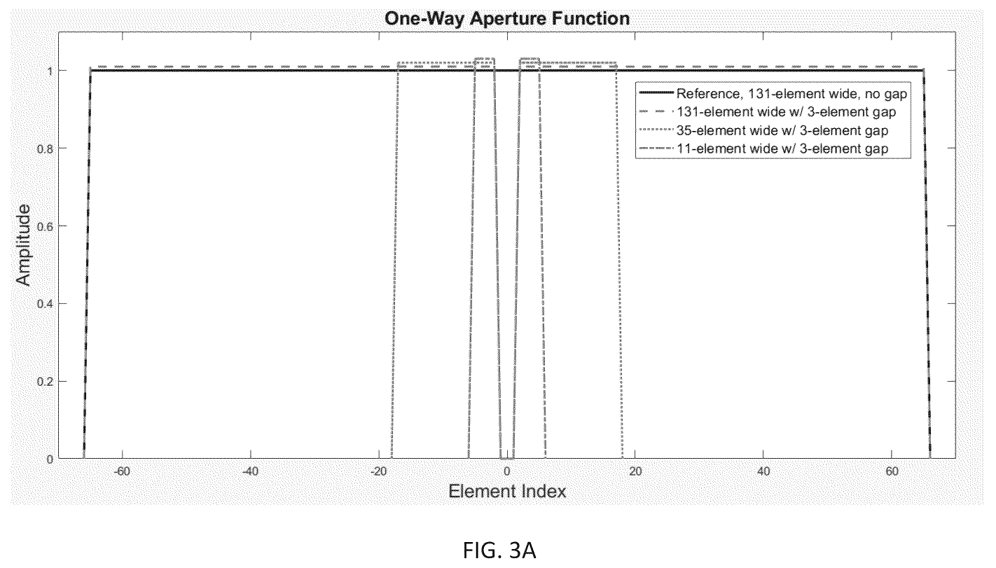

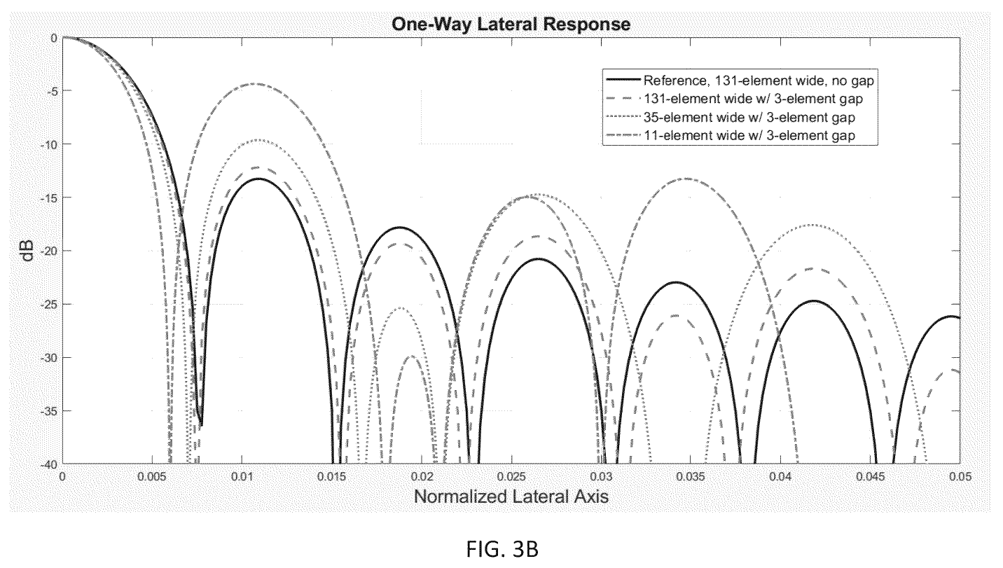

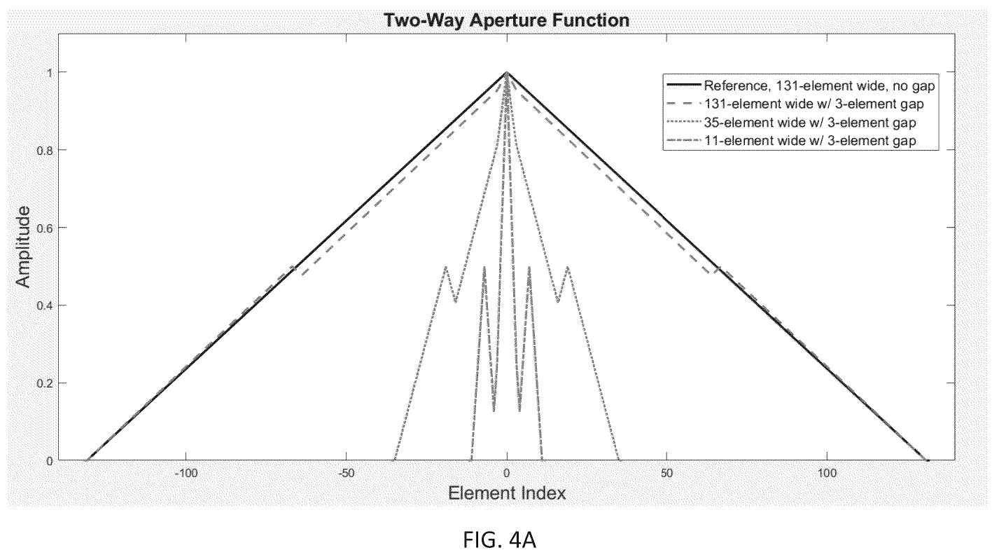

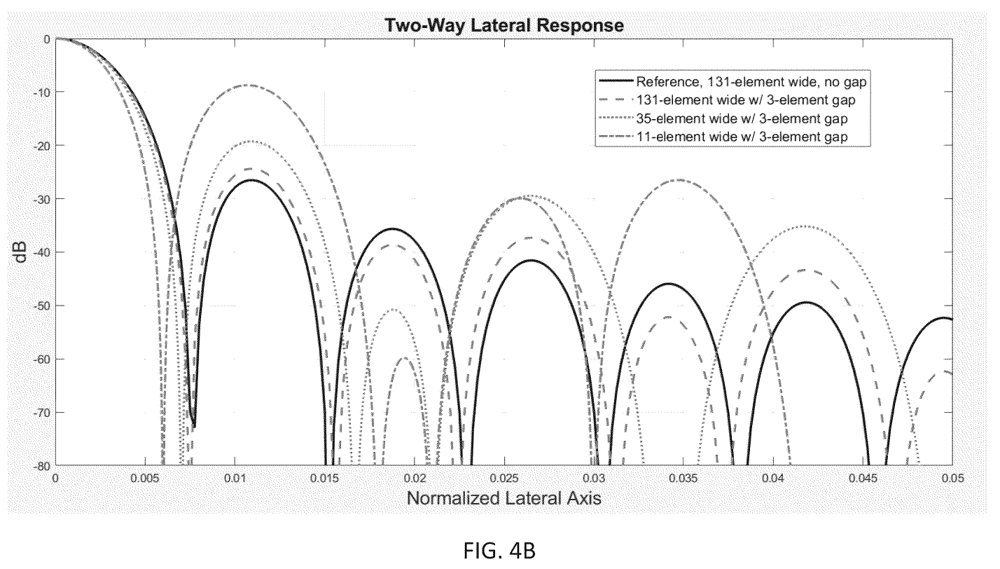

FIGS. 3A, 3B, 4A, and 4B show graphs of a one-way aperture function (FIG. 3A) when a gap between transducer assemblies is in the middle of the active transmit or receive aperture, the respective one-way lateral response (FIG. 3B), a two-way (round-trip) aperture function (FIG. 4A), when the gap is in the middle of both transmit and receive apertures, and the respective two-way lateral response (FIG. 4B) of an exemplary 131-ultrasound transducer assembly configuration with no gaps (represented by the unbroken line), a 131-ultrasound transducer assembly configuration with a three-element gap (represented by the spaced apart broken line), a 35-ultrasound transducer assembly configuration with a three element gap (represented by the dotted line), and a 11-ultrasound transducer assembly configuration with a three element gap (represented by the closely packed broken line). The aperture functions are plotted staggered in amplitude for a given gap width but for different active aperture widths representing different choices of f-number or focal depth. The one-way lateral response represents performance of dynamic receive focusing at depths away from the transmit focus, while the two-way (con-focal) response represents performance at the transmit focus depth assuming receive focusing. The lateral axis for the lateral response plots here is scaled by the aperture widths to match the beamwidths of different aperture width cases, making it easier to compare the effect on side lobe levels. As the ratio of aperture width to gap width decreases, the side lobes increase from about −13 dB to about −4 dB for one-way and about −26 dB to about −8 dB for two-way responses, as the aperture shrinks to several elements around the gap (shallow depth imaging). A gap in middle of the active transmit and/or receive aperture may present an undesirable scenario, wherein, as the active aperture centroid (beam origin) moves away from the gap the side lobes approach and finally match that of the reference when the active aperture no longer includes the gap. In some embodiments, as shown in the curved one-dimensional array configuration 525 in FIG. 2, if the ultrasound transducer assemblies are non-coplanar, the coherent (phase-sensitive) sum of their output beams degrades the focus, reduces lateral resolution and sensitivity, and increases acoustic clutter.

As such, provided herein are methods and systems to reduce side lobes caused by inter-ultrasound transducer assembly space, to attain coherence across and among the non-coplanar ultrasound transducer assemblies.

In some embodiments, the methods and systems herein employ an input parameter to an on-chip delay and aperture weight (apodization) computer of the delay and weight computer that extends beyond the individual ultrasound transducer assembly's boundaries. In some embodiments, the digital channel data from the edge columns (rows) of adjacent matrix configuration arrays are interpolated to create synthetic data for the missing columns (rows) in the inter-ultrasound transducer assembly space. In some embodiments, the received and synthesized channel data then are delayed and summed to form receive beams. In some embodiments, the inter column (row) interpolation of the digital channel data can be a nearest neighbor, linear, a cubic, or any combination thereof. The interpolation and beam formation over the missing columns can be taken care of by the respective ultrasound transducer assemblies or by an external processor and added to the matrix configuration outputs.

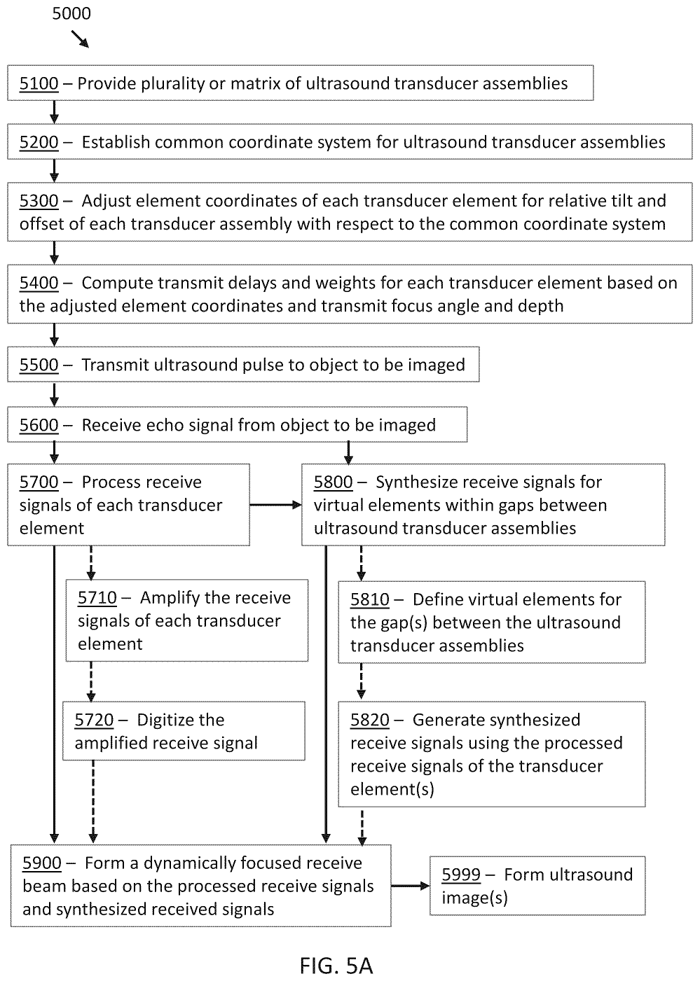

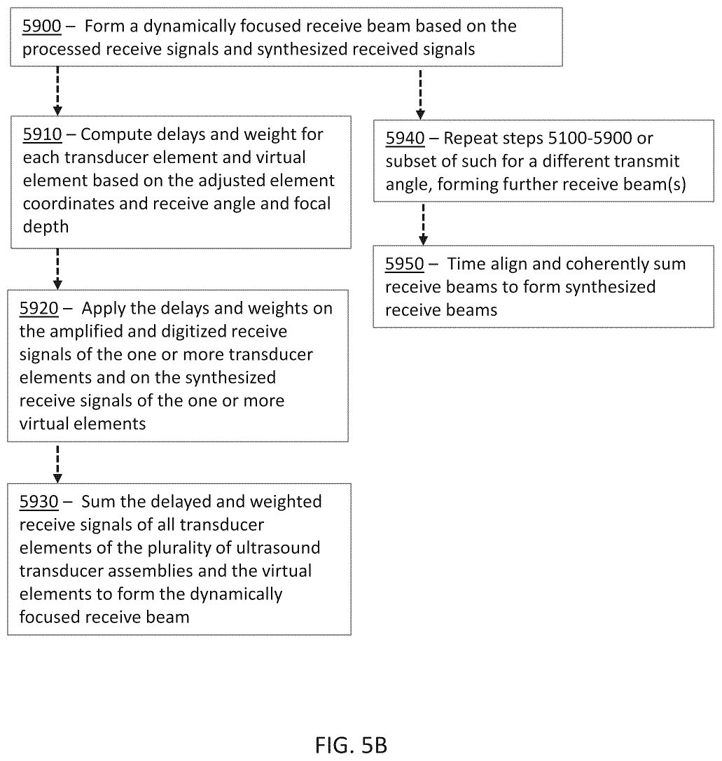

FIG. 5A shows a flow chart of an exemplary method 5000 of ultrasound beam forming and imaging with a plurality of ultrasound transducer assemblies. In a step S100, a plurality or matrix of ultrasound transducer assemblies as described herein may be provided. As described herein, each ultrasound transducer assembly may comprise a plurality of transducer elements. In a step S200, a common coordinate system may be established for the ultrasound transducer assemblies. In a step S300, element coordinates of each transducer element may be adjusted for the relative tilt and offset of each transducer assembly with respect to the common coordinate system. In a step S400, transmit delays and weights for each transducer element may be computed based on the adjusted element coordinates and transmit focus angle and depth. In a step S500, an ultrasound pulse may be transmitted to an object to be imaged using the plurality or matrix of ultrasound transducer assemblies. In a step S600, an echo signal may be received from the object. In a step S700, receive signals of each transducer element may be processed. In a step S800, receive signals for one or more virtual elements within gaps between the ultrasound transducer assemblies may be synthesized. In a step S900, a dynamically focused receive beam may be formed based on the processed receive signals of the one or more transducer elements and synthesized receive signals of the one or more virtual elements. In a step S999, based on the receive beam, one or more ultrasound images may be formed. These image(s) may be two-dimensional (2D) or three-dimensional (3D).

Referring back to the step S700, the receive signals of each transducer element may be processed by a step S710 of amplifying the receive signals of each transducer element and a step S720 of digitizing the amplified receive signal of each transducer element.

Referring back to the step S800, receive signals for the virtual elements may be synthesized by a step S810 of defining virtual elements for the one or more gaps between the ultrasound transducer assemblies and a step S820 of generating the synthesized receive signals for the virtual elements using the processed receive signals of the one or more transducer elements. In some embodiments, generating the synthesized receive signal of an individual virtual element comprises identifying a nearest transducer element to said individual virtual element and assigning the processed receive signal from said individual element as the synthesized receive signal of said individual virtual element. In some embodiments, generating the synthesized receive signal of an individual virtual element comprises identifying a first nearest transducer element on a first ultrasound transducer assembly on a first side of an individual gap, identifying a second nearest transducer element on a second transducer assembly on a second side of the individual gap opposite the first side, generating a linear interpolation of the processed receive signals of the first and second nearest transducer elements based on their distances to the virtual element, and assigning said linear interpolation as the synthesized receive signal of said individual virtual element.

FIG. 5B shows a flow chart of an exemplary break-down of the step S900. In the step S900, the dynamically focused receive beam may be formed by a step S910 of computing delays and weight for each transducer element and virtual element based on the adjusted transducer and virtual element coordinates and receive angle and focal depth, a step S920 of applying the delays and weights on the amplified and digitized receive signals of the one or more transducer elements and on the synthesized receive signals of the one or more virtual elements, and a step S930 of summing the delayed and weighted receive signals of all transducer elements of the plurality of ultrasound transducer assemblies and the virtual elements to form the dynamically focused receive beam.

The above technique(s) can lower the side lobes due to the discontinuities (gaps) in the receive aperture function, but side lobes due to discontinuities in the transmit aperture function can still remain. In some embodiments, many of the steps of method 5000 may be repeated for the same receive beam but for a different transmit angle and/or from a different transmit focus, in a step S940, and the receive beams formed in response to spatially distinct transmit beams may be time aligned and coherently summed, in a step S950, to form synthesized receive beams. This technique is commonly referred to as dynamic transmit focusing or retrospective transmit focusing and is described in U.S. Pat. Nos. 8,241,216 and 8,690,781, which are incorporated herein by reference. This technique can be used to improve focusing away from the static transmit focus depth of conventional beamforming. In many embodiments, it amounts to transmit aperture synthesis where a wide continuous coherent transmit aperture is synthesized for all depths along the receive line of sight (beam) from narrower coherent (i.e., stationary phase) segments of a set of transmit beams with laterally distinct foci (e.g., distinct insonification angles). Given a transmit beam, the segment of the (static) transmit aperture function that coherently contributes to a particular receive beam sample may be centered at the intersection of the transmit aperture and the line that connects the transmit focus and receive sample. Therefore, it can vary as a function of the receive focus depth and angle. Note that in these conventional techniques, the contributing transmit beams can have continuous aperture functions. In many embodiments here, the transmit beams with coherent segments that fall into the aperture gap are excluded from the transmit aperture synthesis.

Although the above steps show method 5000 in accordance with many embodiments, a person of ordinary skill in the art will recognize many variations based on the teaching described herein. The steps may be completed in a different order. Steps may be added or deleted. Some of the steps may comprise sub-steps. Many of the steps may be repeated as often as advantageous. Many of the steps may be performed by processing circuitry.

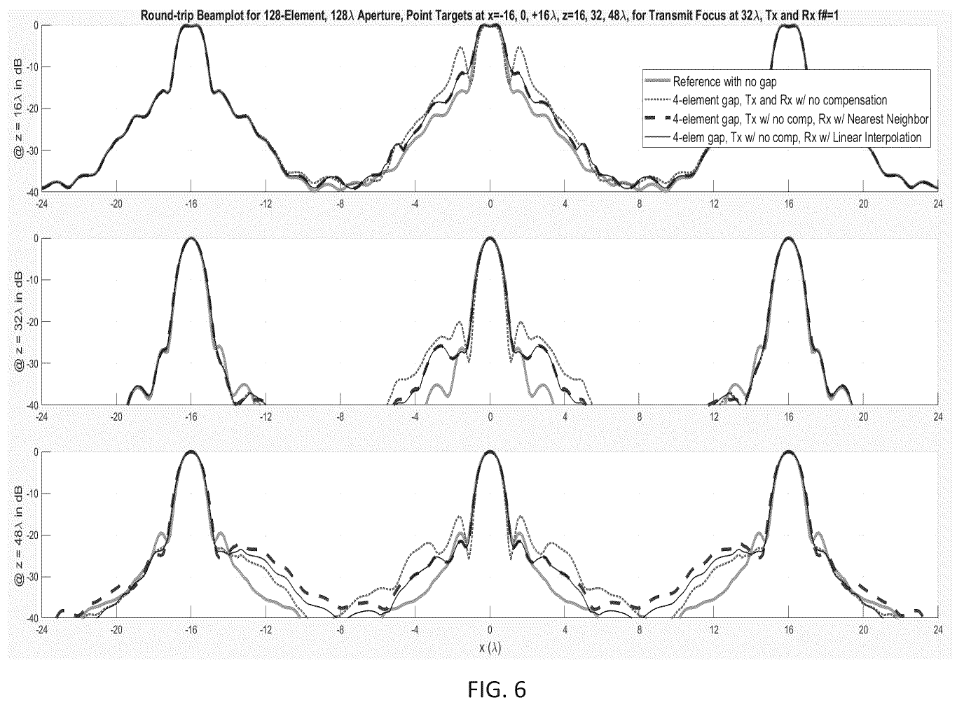

As examples of the implemented method and comparative results, FIG. 6 shows a graph of the lateral response of a 128-element array with λ pitch (i.e., 128λ aperture) with 4 missing elements in the middle. Therein, the transmit beam is focused at 32λ depth with an f-number of 2, and the receive beam is dynamically focused with f-number of 1. A 3×3 grid of pin targets are at 16, 32 and 48λ depths across a −16λ to 16λ span in azimuth with a uniform 16λ spacing. The plots in thick, unbroken lines are the lateral response of a reference array with no gap in the middle. The plots in thin, broken lines are that of an array with a gap but no gap compensation. For the other two cases, the gap in the transmit aperture is not compensated. The gap in the receive aperture is compensated by first creating synthetic channel data for the virtual elements in the gap, and then applying delay on the synthetic data using the coordinates for the virtual elements. The synthetic channel data is created by duplicating the receive signal of the nearest neighbor elements (plots in thick, broken lines) and by linear interpolation of the receive signals of the elements on either side of the gap (plots in thin, unbroken lines). Note the increased side lobes for the targets in the center due to the gap in the middle.

In some embodiments, the (x, y, z) coordinates of each pixel of the matrix array is programmable and the programmed coordinates are inputs to the delay computer of the transducer assembly. Any tilt that is deterministic (e.g., ones that are introduced during manufacturing) then may be compensated for by an input parameter generator before they are communicated to the transducer assembly. Tilts that vary with time or with use (e.g., a multi-transducer assembly patch on a flexible substrate) can be compensated for by an adaptive focusing algorithm that varies the planar tilt estimates until the coherent sum of the matrix of DISCs is maximized.

The methods descried herein can also be used to create an incoherent matrix of transducer assemblies that are aligned for spatial compounding as well.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater than or less than the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for ultrasound beam forming and imaging with a plurality of ultrasound transducer assemblies, each ultrasound transducer assembly comprising a plurality of transducer elements, the method comprising: (i) adjusting element coordinates of each transducer element for relative tilt and offset of each transducer assembly with respect to a common coordinate system; (ii) computing transmit delays and weights for each transducer element based on the adjusted element coordinates and transmit focus angle and depth; (iii) transmitting a pulse and receiving echo from an object being imaged; (iv) processing receive signals of each transducer element; (v) synthesizing receive signals for one or more virtual elements within gaps between the ultrasound transducer assemblies; and (vi) forming a dynamically focused receive beam based on the processed receive signals of the one or more transducer elements and synthesized receive signals of the one or more virtual elements.

Clause 2. The method of Clause 1, wherein step (iv) comprises: (a) amplifying the receive signals of each transducer element, and (b) digitizing the amplified receive signal of each transducer element.

Clause 3. The method of Clause 1 or 2, wherein step (v) comprises: (a) defining virtual elements for the one or more gaps between the ultrasound transducer assemblies, and (b) generating the synthesized receive signals for the virtual elements using the processed receive signals of the one or more transducer elements.

Clause 4. The method of Clause 3, wherein generating the synthesized receive signal of an individual virtual element comprises identifying a nearest transducer element to said individual virtual element and assigning the processed receive signal from said individual element as the synthesized receive signal of said individual virtual element.

Clause 5. The method of Clause 3 or 4, wherein generating the synthesized receive signal of an individual virtual element comprises identifying a first nearest transducer element on a first ultrasound transducer assembly on a first side of an individual gap, identifying a second nearest transducer element on a second transducer assembly on a second side of the individual gap opposite the first side, generating a linear interpolation of the processed receive signals of the first and second nearest transducer elements, and assigning said linear interpolation as the synthesized receive signal of said individual virtual element.

Clause 6. The method of any one of Clauses 1-5, wherein step (vi) comprises: (a) computing delays and weight for each transducer element and virtual element based on the adjusted element coordinates and receive angle and focal depth, (b) applying the delays and weights on the amplified and digitized receive signals of the one or more transducer elements and on the synthesized receive signals of the one or more virtual elements, and (c) summing the delayed and weighted receive signals of all transducer elements of the plurality of ultrasound transducer assemblies and the virtual elements to form the dynamically focused receive beam.

Clause 7. The method of any one of Clauses 1-6, wherein the steps (iv) to (vi) are repeated for a receive beam line of sight but using the echo received in response to a plurality of transmit beams with foci that are laterally distinct, and wherein the receive beams formed are time aligned and coherently summed to form synthesized receive beams.

Clause 8. The method of any one of Clauses 1-7, wherein an application specific integrated circuit (ASIC) is integrated with at least one ultrasound transducer assembly, and wherein the ASIC performs one or more of steps (i) to (vi) to form the dynamically focused receive beam.

Clause 9. The method of any one of Clauses 1-8, wherein at least one ultrasound transducer assembly of the plurality is comprised of one or more capacitive micromachined ultrasound transducer (cMUT), piezoelectric micromachined ultrasound transducer (pMUT), or bulk PZT transducer elements.

Clause 10. The method of any one of Clauses 1-9, wherein the plurality of ultrasound transducer assemblies comprises a matrix or array of the ultrasound transducer assemblies.

Clause 11. The method of Clause 10, wherein the matrix or array of the ultrasound transducer assemblies comprises a 1-dimensional array, a 2-dimensional matrix, a curved matrix or array, a piece-wise curved matrix or array, or a flat matrix or array of the ultrasound transducer assemblies.

Clause 12. The method of any one of Clauses 1-11, wherein the plurality of the transducer elements for at least one ultrasound transducer assembly comprises a 2-dimensional matrix of the transducer elements.

Clause 13. The method of any one of Clauses 1-12, further comprising providing the plurality of ultrasound transducer assemblies on a wearable device.

Clause 14. A method of imaging a target object, the method comprising: using an imaging device to generate an image of the target object, wherein the imaging device comprises a plurality of ultrasound transducer assemblies and control circuitry operatively coupled thereto, and wherein the control circuitry is configured to operate the plurality of the ultrasound transducer assemblies according to the method of any one of Clauses 1-13.

Clause 15. A method of imaging a target object, the method comprising: providing an imaging device to generate an image of the target object, wherein the imaging device comprises a plurality of ultrasound transducer assemblies and control circuitry operatively coupled thereto, and wherein the control circuitry is configured to operate the plurality of the ultrasound transducer assemblies according to the method of any one of Clauses 1-14.

Clause 16. A method of imaging a target object, the method comprising: providing a plurality of ultrasound transducer assemblies, each ultrasound transducer assembly comprising a plurality of ultrasound transducer elements; tiling the plurality of ultrasound transducer assemblies into a matrix configuration; and acquiring an image of the target object using the tiled plurality of ultrasound transducer assemblies, wherein the plurality of ultrasound transducer assemblies is operatively coupled to control circuitry configured to operate the plurality of ultrasound transducer assemblies according to the method of any one of Clauses 1-15.

Clause 17. The method of Clause 16, wherein tiling the plurality of ultrasound transducer assemblies comprises arranging the plurality of ultrasound transducer assemblies into a 1-dimensional array, a 2-dimensional matrix or array, a curved matrix or array, a piece-wise curved matrix or array, or a flat matrix or array.

Clause 18. A system for imaging a target object, the system comprising: a plurality of ultrasound transducer assemblies, each ultrasound transducer assembly comprising a plurality of transducer elements; and control circuitry operatively coupled to the plurality of ultrasound transducer assemblies and configured to operate the plurality of ultrasound transducer assemblies according to the method of any one of Clauses 1-17.

Clause 19. The system of Clause 18, wherein the ultrasound transducer assemblies are tileable into a matrix configuration.

Clause 20. The system of Clause 19, wherein the matrix configuration is a 1-dimensional array, a 2-dimensional matrix or array, a curved matrix or array, a piece-wise curved matrix or array, or a flat matrix or array.

Clause 21. A method of imaging a target object, the method comprising: providing a plurality of ultrasound transducer assemblies, each ultrasound transducer assembly comprising a plurality of ultrasound transducer elements; tiling the plurality of ultrasound transducer assemblies into a matrix or array configuration; and acquiring an image of the target object using the tiled plurality of ultrasound transducer assemblies.

Clause 22. The method of Clause 21, wherein the matrix or array configuration is a 1-dimensional array, a 2-dimensional matrix or array, a curved matrix array, a piece-wise curved matrix or array, or a flat matrix or array.

Clause 23. The method of Clause 21 or 22, wherein each ultrasound transducer assembly further comprises an application specific integrated circuit (ASIC) integrated thereon.

Clause 24. The method of any one of Clauses 21-23, wherein each ultrasound transducer assembly of the plurality is adjusted for relative tilt and offset with respect to a common coordinate system for the plurality of ultrasound transducer assemblies.

Clause 25. A system for imaging a target object, the system comprising: (a) a plurality of ultrasound transducer assemblies, each ultrasound transducer assembly comprising a plurality of transducer elements; and (b) control circuitry operatively coupled to the plurality of ultrasound transducer assemblies and configured to operate the plurality of ultrasound transducer assemblies, wherein the ultrasound transducer assemblies are tileable into a matrix or array configuration.

Clause 26. The system of Clause 25, wherein the matrix or array configuration is a 1-dimensional array, a 2-dimensional matrix or array, a curved matrix or array, a piece-wise curved matrix or array, or a flat matrix or array.

Clause 27. The system of Clause 25 or 26, wherein one or more gaps are present between adjacent ultrasound transducer assemblies when tiled into the matrix or array configuration.

Clause 28. The system of any one of Clauses 25-27, wherein each ultrasound transducer assembly comprises an application specific integrated circuit (ASIC) operatively coupled to and integrated with the plurality of transducer assemblies for each ultrasound transducer assembly.

Clause 29. The system of any one of Clauses 25-28, wherein each ultrasound transducer assembly of the plurality is adjusted for relative tilt and offset with respect to a common coordinate system for the plurality of ultrasound transducer assemblies.

Clause 30. The system of any one of Clauses 25-29, wherein at least one ultrasound transducer assembly of the plurality is comprised of one or more capacitive micromachined ultrasound transducer (cMUT), piezoelectric micromachined ultrasound transducer (pMUT), or bulk PZT transducer elements.

Clause 31. The system of any one of Clauses 25-30, wherein the plurality of the transducer elements for at least one ultrasound transducer assembly comprises a matrix or array of the transducer elements.

Clause 32. The system of any one of Clauses 25-31, wherein the plurality of the transducer elements for at least one ultrasound transducer assembly comprises a 2-dimensional matrix of the transducer elements.

Clause 33. The system of any one of Clauses 25-32, further comprising a wearable housing configured to hold the plurality of ultrasound transducer assemblies in the matrix or array configuration.

Clause 34. The system of Clause 33, wherein the wearable housing is a patch or band.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A method for ultrasound beam forming and imaging with a plurality of ultrasound transducer assemblies, each ultrasound transducer assembly comprising a plurality of transducer elements, the method comprising:
(i) adjusting element coordinates of each transducer element for relative tilt and offset of each transducer assembly with respect to a common coordinate system;
(ii) computing transmit delays and weights for each transducer element based on the adjusted element coordinates and transmit focus angle and depth;
(iii) transmitting a pulse and receiving echo from an object being imaged;
(iv) processing receive signals of each transducer element;
(v) synthesizing receive signals for one or more virtual elements within gaps between the ultrasound transducer assemblies; and
(vi) forming a dynamically focused receive beam based on the processed receive signals of the one or more transducer elements and synthesized receive signals of the one or more virtual elements.

2. The method of claim 1, wherein step (iv) comprises:
amplifying the receive signals of each transducer element, and
digitizing the amplified receive signal of each transducer element.

3. The method of claim 1, wherein step (v) comprises:
defining virtual elements for the one or more gaps between the ultrasound transducer assemblies, and
generating the synthesized receive signals for the virtual elements using the processed receive signals of the one or more transducer elements.

4. The method of claim 3, wherein generating the synthesized receive signal of an individual virtual element comprises identifying a nearest transducer element to said individual virtual element and assigning the processed receive signal from said individual element as the synthesized receive signal of said individual virtual element.

5. The method of claim 3, wherein generating the synthesized receive signal of an individual virtual element comprises identifying a first nearest transducer element on a first ultrasound transducer assembly on a first side of an individual gap, identifying a second nearest transducer element on a second transducer assembly on a second side of the individual gap opposite the first side, generating a linear interpolation of the processed receive signals of the first and second nearest transducer elements, and assigning said linear interpolation as the synthesized receive signal of said individual virtual element.

6. The method of claim 1, wherein step (vi) comprises:
computing delays and weight for each transducer element and virtual element based on the adjusted element coordinates and receive angle and focal depth,
applying the delays and weights on the amplified and digitized receive signals of the one or more transducer elements and on the synthesized receive signals of the one or more virtual elements, and
summing the delayed and weighted receive signals of all transducer elements of the plurality of ultrasound transducer assemblies and the virtual elements to form the dynamically focused receive beam.

7. The method of claim 1, wherein the steps (iv) to (vi) are repeated for a receive beam line of sight but using the echo received in response to a plurality of transmit beams with foci that are laterally distinct, and wherein the receive beams formed are time aligned and coherently summed to form synthesized receive beams.

8. The method of claim 1, wherein an application specific integrated circuit (ASIC) is integrated with at least one ultrasound transducer assembly, and wherein the ASIC performs one or more of steps (i) to (vi) to form the dynamically focused receive beam.

9. A method of imaging a target object, the method comprising: providing a plurality of ultrasound transducer assemblies, each ultrasound transducer assembly comprising a plurality of ultrasound transducer elements; tiling the plurality of ultrasound transducer assemblies into a matrix or array configuration; and acquiring an image of the target object using the tiled plurality of ultrasound transducer assemblies.

10. The method of claim 9, wherein the matrix or array configuration is a 1-dimensional array, a 2-dimensional matrix or array, a curved matrix array, a piece-wise curved matrix or array, or a flat matrix or array.

11. The method of claim 9, wherein each ultrasound transducer assembly further comprises an application specific integrated circuit (ASIC) integrated thereon.

12. The method of claim 9, wherein each ultrasound transducer assembly of the plurality is adjusted for relative tilt and offset with respect to a common coordinate system for the plurality of ultrasound transducer assemblies.

13. A system for imaging a target object, the system comprising:
a plurality of ultrasound transducer assemblies, each ultrasound transducer assembly comprising a plurality of transducer elements; and
control circuitry operatively coupled to the plurality of ultrasound transducer assemblies and configured to operate the plurality of ultrasound transducer assemblies,
wherein the ultrasound transducer assemblies are tileable into a matrix or array configuration.

14. The system of claim 13, wherein the matrix or array configuration is a 1-dimensional array, a 2-dimensional matrix or array, a curved matrix or array, a piece-wise curved matrix or array, or a flat matrix or array.

15. The system of claim 13, wherein one or more gaps are present between adjacent ultrasound transducer assemblies when tiled into the matrix or array configuration.

16. The system of claim 13, wherein each ultrasound transducer assembly comprises an application specific integrated circuit (ASIC) operatively coupled to and integrated with the plurality of transducer assemblies for each ultrasound transducer assembly.

17. The system of claim 13, wherein each ultrasound transducer assembly of the plurality is adjusted for relative tilt and offset with respect to a common coordinate system for the plurality of ultrasound transducer assemblies.

18. The system of claim 13, wherein at least one ultrasound transducer assembly of the plurality is comprised of one or more capacitive micromachined ultrasound transducer (cMUT), piezoelectric micromachined ultrasound transducer (pMUT), or bulk PZT transducer elements.

19. The system of claim 13, wherein the plurality of the transducer elements for at least one ultrasound transducer assembly comprises a matrix or array of the transducer elements.

20. The system of claim 13, wherein the plurality of the transducer elements for at least one ultrasound transducer assembly comprises a 2-dimensional matrix of the transducer elements.
